(19) United States
(12) Patent Application Publication
(10) Pub. No.: US 2008/0271211 A1
(43) Pub. Date: Oct. 30, 2008

(54) Materials and Methods for Providing Resistance to Plant Pathogens in Non-Transgenic Plant Tissue

(76) Inventors: Jane E. Polston, Gainesville, FL (US); Ernest Hiebert, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/628,101

(22) PCT Filed: May 27, 2005
(86) PCT No.: PCT/US2005/018750

§ 371 Date: Dec. 12, 2007

Related U.S. Application Data
(60) Provisional application No. 60/575,485, filed on May 28, 2004.

(51) Int. Cl. A01H 5/00

(52) U.S. Cl. 800/301; 800/302

(57) ABSTRACT

The subject invention concerns materials and methods for providing disease and pathogen resistance to a plant. Transformed or transgenic rootstock of a plant with genetically engineered resistance to a plant pathogen, such as a viral pathogen, is grafted onto a compatible non-transgenic plant tissue, e.g., a scion compatible with the rootstock. The non-transgenic portion of the grafted plant is provided with resistance to the plant pathogen. The subject invention also concerns a plant comprising a transformed or transgenic rootstock having resistance to a plant pathogen grafted onto a compatible non-transgenic plant tissue.

### CROSS-REFERENCE TO RELATED APPLICATION

This application claims the benefit of U.S. Provisional Application Ser. No. 60/575,485, filed May 28, 2004.

### BACKGROUND OF THE INVENTION

Major losses of agricultural crop yields and quality can result from infection of crop plants by plant pathogens, such as viruses, bacteria, and fungi. Many agricultural crops are susceptible to infection by plant pathogens. For example, viral infections in plants are frequently responsible for growth inhibition, unwanted or undesirable morphological changes, decreased yield, etc. Thus, plant pathogens can seriously damage a crop and reduce its economic value to the grower. This leads to a higher cost for the consumer. While attempts to control or prevent infection of a crop by a plant pathogen have been made, plant pathogens continue to be a significant problem around the world.

In the past decade, scientists have developed means to produce plants that are resistant to infection by a plant pathogen using genetic engineering techniques. Typically, genetic material which provides the plant with resistance to the pathogen is incorporated into the genome of a plant and, therefore, can be passed on to its progeny. Transgenic plants have been produced that are resistant to infection by a viral pathogen through the incorporation and expression of virus-derived genes or gene fragments within the plant. However, many people are still uncomfortable and/or unwilling to consume or utilize transgenic fruits, vegetables, and other crop products. In many countries, transgenic plants are not permitted.

Thus, there remains a need for a way to genetically engineer a plant such that the plant is provided with resistance to a plant pathogen yet wherein the plant product that is eaten or consumed is not transgenic in nature.

### BRIEF SUMMARY OF THE INVENTION

The subject invention concerns materials and methods for providing disease resistance to a plant. Rootstock of a transgenic plant having genetically engineered resistance to a plant pathogen, such as a viral pathogen, is grafted onto a compatible non-transgenic plant tissue, e.g., a scion compatible with the transgenic rootstock. The plant produced has increased or improved resistance to a plant pathogen when compared to a wild type plant or a plant lacking the transgenic rootstock. In one embodiment, plants comprising a transgenic rootstock with engineered resistance to Tomato yellow leaf curl virus (TYLCV) and a scion taken from a non-transgenic, TYLCV-susceptible plant had significantly milder symptoms after inoculation with whiteflies carrying TYLCV than plants which did not have genetically-engineered resistant rootstocks. The subject invention provides methods and materials for capturing genetically-engineered pathogen resistance without making the edible parts of the plant transgenic. The subject invention can be used in a number of different situations where grafting is a normal part of horticultural practices such as tree crops, small fruits, and vegetables.

### BRIEF DESCRIPTION OF THE DRAWING

FIG. 1 shows grafted tomato plants according to the present invention.

### BRIEF DESCRIPTION OF THE SEQUENCES

SEQ ID NO: 1 is a polynucleotide which can be used according to the subject invention.

SEQ ID NO: 2 is a polynucleotide which can be used according to the subject invention.

SEQ ID NO: 3 is a polynucleotide which can be used according to the subject invention.

### DETAILED DISCLOSURE OF THE INVENTION

The subject invention concerns materials and methods for providing increased or improved disease and pathogen resistance to a plant. In one embodiment of the present methods, transformed or transgenic rootstock of a plant with genetically engineered resistance to a plant pathogen, such as a viral pathogen, is grafted onto a compatible non-transgenic plant tissue, e.g., a scion compatible with the rootstock. As used herein, the term “compatible” refers to the ability of the grafted rootstock and plant tissue to grow together and survive. It is well known that compatible rootstock and plant tissue grafts do not have to be from the same plant species. For example, tomato scions can be grafted onto eggplant rootstock. Transgenic rootstock can be prepared using standard methods.

In one embodiment, plant cells are transformed with a polynucleotide or nucleic acid that when incorporated into a plant cell provides or confers increased or improved resistance to one or more plant pathogens for a plant or plant tissue grown from the transformed plant cell. Polynucleotide and nucleic acid constructs that can provide or confer resistance to one or more plant pathogens, e.g., a viral pathogen, are known in the art (see, for example, Zhu et al. (2003); Barajas et al. (2004); Lanfermeijer et al. (2004); and U.S. Pat. Nos. 6,818,804; 6,777,588; 6,750,382; 6,716,967; 6,667,426; 6,172,280; 6,852,907; 6,548,742; 6,057,492; 6,127,601; 5,530,193; and 5,998,699). In one embodiment, the polynucleotide construct comprises a nucleotide sequence that encodes all or a portion of the replication (Rep) protein of a TYLCV and all or a portion of a TYLCV Rep gene intergenic region (IR). The Rep sequences used in the present invention can be from any strain of TYLCV. In one embodiment, the Rep gene sequence of a polynucleotide of the present invention is from TYLCV-Florida (TYLCV-Fl). In one embodiment, a polynucleotide of the invention comprises from about 50 to 100 nucleotides of a Rep gene intergenic region and about 300 to 700 nucleotides of the 5′ terminus of a TYLCV Rep gene. In a further embodiment, a polynucleotide of the invention comprises about 70 to 90 nucleotides of the IR and about 400 to 450 nucleotides of the 5′ terminus of the Rep gene. In a still further embodiment, a polynucleotide of the invention comprises about 80 to 85 nucleotides of the IR and about 410 to about 430 nucleotides of the 5′ terminus of the Rep gene. In an exemplified embodiment, a polynucleotide construct of the invention comprises the nucleotide sequence shown in SEQ ID NO: 1.

In a still further embodiment, a polynucleotide of the invention comprises a first polynucleotide that comprises all or a portion of a Rep gene IR, followed by all or a portion of the Rep gene, operatively linked to a second polynucleotide comprising, in antisense orientation, all or a portion of the Rep gene, followed by all or a portion of a Rep gene IR also in antisense orientation. Optionally, a linker nucleotide sequence can be provided operatively linking the polynucleotides. In one embodiment, the linker sequence can comprise a sequence that is downstream of the 3′ end of the Rep gene. In one embodiment, a polynucleotide of the invention comprises from about 50 to 100 nucleotides, or about 70 to 90 nucleotides, or about 80 to 85 nucleotides, of a Rep gene IR, followed by a complete Rep gene sequence or a fragment thereof of about 300 to 700 nucleotides of the 5′ terminus of the Rep gene, optionally followed by about 10 to 100 nucleotides downstream of the 3′ end of a Rep gene, and/or optionally followed by a linker of between about 1 to 100 nucleotides, and/or optionally followed by a complete Rep gene in antisense orientation or about 300 to 700 nucleotides, or about 500 to 600 nucleotides, of the 5′ terminus of a Rep gene in the antisense orientation and about 50 to 100 nucleotides, or about 80 to 90 nucleotides, of a Rep gene IR also in the antisense orientation. In an exemplified embodiment, a polynucleotide of the invention has the nucleotide sequence shown in SEQ ID NO: 2.

In another embodiment, a polynucleotide of the invention comprises from about 50 to 100 nucleotides of a Rep gene IR and about 300 to 700 nucleotides of the 5′ terminus of a TYLCV Rep gene, wherein the IR and Rep sequences are in the antisense orientation. In a further embodiment, a polynucleotide of the invention comprises from about 80 to 90 nucleotides of a Rep gene IR and about 500 to 600 nucleotides of the 5′ terminus of the Rep gene, both in the antisense orientation. In an exemplified embodiment, a polynucleotide of the invention has the nucleotide sequence shown in SEQ ID NO: 3 (595 nucleotides of the 5′ terminus of the TYLCV Rep gene (nucleotides 2021 to 2615 of GenBank Accession No. AY530931) (encompasses the entire C4 gene) in the antisense orientation, followed by 85 nucleotides of the IR (nucleotides 2616 to 2701) in the antisense orientation). As used herein, the term “antisense” refers to polynucleotides that provide for transcribed sequences that are at least partially complementary to the transcript from genes that are in the normal, sense orientation.

Polynucleotides of the present invention can be introduced directly into plants, such as tomato, by, for example, Agrobacterium-mediated transformation, and transformed and transgenic plant lines prepared therefrom. Transgenic plants can be prepared from transformed plant cell or tissue. Once a pathogen resistant transformed or transgenic plant has been produced, the rootstock can be obtained therefrom and used for grafting in accordance with the present invention. In one embodiment, a transgenic rootstock is grafted to a scion of a non-transgenic plant that is susceptible to infection by the plant pathogen. Grafting can be accomplished using standard materials and methods known in the art. (See, for example, Black et al. (2003); Fernandez-Garcia et al. (2004); Edelstein et al. (2004); see Worldwide Website: www.paramount-seeds.com/Paramountonline/grafting.htm; see Worldwide Website: www.agnet.org/library/article/eb480.html).

In an exemplified embodiment, a transgenic rootstock with engineered resistance to Tomato yellow leaf curl virus (TYLCV) is grafted to a scion taken from a non-transgenic, TYLCV-susceptible plant. The grafted plants exhibited significantly milder symptoms after inoculation with whiteflies carrying TYLCV than plants which did not have genetically-engineered resistant rootstocks. This is due to the eliciting of a resistance factor in the transgenic rootstocks that can be moved into the scions and reverse the infection that occurs in the scions after inoculation. Though it is known in the art that transgenic plants with virus resistance have a mobile translocatable factor, it has not been demonstrated that this facility could be exploited to produce virus-resistant crops by coupling it with horticultural grafting to provide a plant that produces pathogen resistant fruit that are not genetically-engineered.

The present invention also concerns plants comprising a transgenic rootstock having genetically engineered resistance to a plant pathogen and, grafted onto the transgenic rootstock, a non-transgenic plant tissue, such as a scion, that is compatible with the rootstock. In one embodiment, a TYLCV-resistant plant is provided comprising a transgenic rootstock with engineered resistance to Tomato yellow leaf curl virus (TYLCV) and a scion taken from a non-transgenic, TYLCV-susceptible plant. The subject invention also concerns non-transgenic fruit produced by a pathogen resistant plant of the invention.

Plants within the scope of the present invention also include dicotyledonous plants, such as, for example, peas, alfalfa, tomato, tomatillo, melon, chickpea, chicory, clover, kale, lentil, soybean, tobacco, potato, sweet potato, radish, cabbage, rape, apple trees, grape, cotton, sunflower, citrus (including orange, mandarin, kumquat, lemon, lime, grapefruit, tangerine, tangelo, citron, and pomelo), pepper, bean, and lettuce. Plants within the scope of the present invention also include conifers.

Examples of tomato rootstock that can be used to prepare pathogen resistant transgenic rootstock includes, but is not limited to, “PG3” and “Beaufort.” Examples of tomato cultivars that can be used to provide scions for the present invention include, but are not limited to, “Monroe,” “Belle,” Summer Set,” “Match,” Trust,” “Better Boy,” “Celebrity,” “Grace,” “Heinz 1439,” “Roma,” “Rugers,” “Ultra Girl,” “2710,” “BHN 665,” “STM 0227,” “STM 5206,” “Boy oh Boy,” “Jubilation,” “Sunchief,” and “Fabulous.”

The present invention can be used to provide resistance against any plant pathogen for which a pathogen resistant rootstock is available or can be prepared presently or prospectively. The present invention can provide resistance against plant pathogens that include, but are not limited to, viruses or viroids, bacteria, insects, fungi, and the like. Viruses include tobacco or cucumber mosaic virus, ringspot virus, necrosis virus, maize dwarf mosaic virus, tomato yellow leaf curl virus, tomato mottle virus, soybean mosaic virus, tomato spotted wilt virus, barley yellow dwarf virus, citrus tristeza virus (CTV), citrus mosaic virus (CiMV) etc. Bacteria include Xanthomonas axonopodis pv. citri, the pathogen that causes citrus canker.

As used herein, the terms “nucleic acid” and “polynucleotide” refer to a deoxyribonucleotide, ribonucleotide or a mixed deoxyribonucleotide and ribonucleotide polymer in either single- or double-stranded form, and unless otherwise limited, would encompass known analogs of natural nucleotides that can function in a similar manner as naturally-occurring nucleotides. The polynucleotide sequences include both the DNA strand sequence that is transcribed into RNA and the RNA sequence that is translated into protein. The complementary sequence of any nucleic acid or polynucleotide of the present invention is also contemplated within the scope of the invention. It is understood that a particular polynucleotide sequence includes the degenerate codons of the native sequence or sequences which may be introduced to provide codon preference in a specific host cell.

Polynucleotides of the present invention can be composed of either RNA or DNA. Preferably, the polynucleotides are composed of DNA. The subject invention also encompasses those polynucleotides that are complementary in sequence to the polynucleotides disclosed herein. Polynucleotides of the invention can be provided in purified or isolated form.

Because of the degeneracy of the genetic code, a variety of different polynucleotide sequences can encode a polypeptide. A table showing all possible triplet codons (and where U also stands for T) and the amino acid encoded by each codon is described in Lewin (1985). In addition, it is well within the skill of a person trained in the art to create alternative polynucleotide sequences encoding the same, or essentially the same, polypeptides. These degenerate variant and alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides.

The subject invention also concerns variants of the polynucleotides of the present invention. Variant sequences include those sequences wherein one or more nucleotides of the sequence have been substituted, deleted, and/or inserted. The nucleotides that can be substituted for natural nucleotides of DNA have a base moiety that can include, but is not limited to, inosine, 5-fluorouracil, 5-bromouracil, hypoxanthine, 1-methylguanine, 5-methylcytosine, and tritylated bases. The sugar moiety of the nucleotide in a sequence can also be modified and includes, but is not limited to, arabinose, xylulose, and hexose. In addition, the adenine, cytosine, guanine, thymine, and uracil bases of the nucleotides can be modified with acetyl, methyl, and/or thio groups. Sequences containing nucleotide substitutions, deletions, and/or insertions can be prepared and tested using standard techniques known in the art.

Polynucleotides of the subject invention can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules having sequences which are sufficiently homologous with the sequences exemplified herein so as to permit hybridization with that sequence under standard stringent conditions and standard methods (Maniatis, T. et al., 1982). As used herein, “stringent” conditions for hybridization refers to conditions wherein hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature is described by the following formula (Beltz, G. A. et al., 1983):

Tm=81.5 C+16.6 Log[Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

The following is a summary of a series of experiments concerning the subject invention.

A. Resistant transgenic plants were immune to infection by TYLCV. This was demonstrated by the lack of infection in non-inoculated susceptible tissues which had been grafted onto inoculated resistant transgenic plants. Viral DNA was only detected in susceptible non-transgenic tissues and not in resistant transgenic tissues (Table 1).

B. Transgenic plants could not be inoculated by grafting of infected susceptible scions or stocks (Table 2). No to very mild symptoms were seen in transgenic plants, and DNA could be detected in transgenic scions after grafting onto infected susceptible stocks. However, this DNA is not the result of virus replication in the transgenic scions, because after cutting the scion from the plant the DNA could not be detected several weeks later. Hence the DNA is only circulating in the transgenic scions. So detection of DNA by PCR or any other method is not a reliable method for determining resistance in some combinations of grafted plants. That leaves evaluation of symptoms as the best indicator. Since symptom severity has been shown to be related to virus titer and reductions in yield, this is an acceptable method for evaluation of the impacts of trying to impart the resistance factor from transgenic plants to susceptible plants through means of grafting.

C. A resistance factor was translocated and was observed to reduce symptoms in infected susceptible tissues. Symptoms in susceptible infected scions grafted were significantly reduced after grafting to non-inoculated resistant transgenic stocks. Mean symptoms were rated 1.5 which is much lower than the rating of 3.3 on susceptible infected grafted onto non-inoculated susceptible stocks. This resistance factor moved from scion to stock and from stock to scion. (Table 2).

D. Transgenic resistance could overcome an already established infection. Symptom expression in infected susceptible plants was significantly reduced after grafting onto challenged transgenic stocks (Table 4).

E. Susceptible scions became infected after inoculation but within weeks symptom severity began to lessen. Presumably this was due to the turning on of the resistance factor in the stock, which was then translocated to the scions where the established infection was turned off. These results are consistent with results of previous studies (A through D) (Table 5). Not all transgenic plants acted the same; the 45-10 line was superior to line 67-10 in its ability to reduce symptoms in susceptible scions. This suggests that screening transgenic plants for those that function the best for the purposes of transmission of a resistance to susceptible scions and that the effect of the transgenic stock could be improved upon. If resistance was uniform among all the plants in each line, the effects of the transgenic stocks would be more pronounced.

All patents, patent applications, provisional applications, and publications referred to or cited herein are incorporated by reference in their entirety, including all figures and tables, to the extent they are not inconsistent with the explicit teachings of this specification.

### Materials and Methods

### Non-Transgenic Tomato Plants.

Fla. 7613, an advanced breeding line from the breeding program of J. W. Scott, was transformed with 2/5 TYLCV Rep construct. Fla. 7613 is used as a negative control in all the experiments. The 2/5 TYLCV Rep construct has been described in Yang et al. (2004). In one embodiment, a 2/5 TYLCV Rep construct comprises the nucleotide sequence shown in SEQ ID NO: 1 (SEQ ID NO: 1 has 80 nucleotides of the IR (nucleotides 2696 to 2616 of GenBank accession no. AY530931) plus 426 nucleotides of the 5′ terminus of the TYLCV Rep gene).

### Transgenic Tomato Plants.

R₂ generation plants of three 2/5 TYLCV Rep-transformed lines (designated herein as 02-09, 45-10, and 67-10) were used in these studies. Each of these lines arose from unique transformation events. Line 45-10 was shown to have a single copy of the transgene, 67-10 to have two copies of the transgene on the same chromosome, although there was still segregation in both lines for the transgene and for resistance. Greenhouse inoculation studies indicated that the frequency of resistance in line 45-10 was 70% (100% in plants with the gene) and the frequency of resistance was 96.8% in line 67-10. The presence of the transgene was confirmed by PCR in all transgenic plants used in the grafting experiments.

### Graft Transmission.

Before grafting, the presence of the transgene was confirmed by PCR in all transgenic plants used in the grafting experiments. Scions were grafted onto stocks in several studies using the following approach. Plants that were of similar size and had medium flexibility (not too rigid and not too soft) in the tissue were used to graft. The scion and stock were cut at an angle using a sterile scalpel and grafted together keeping the same orientation to line up the xylem and phloem. Parafilm was used to hold the scion against the stock. The scion was covered with a plastic bag and the bag tied around the stock, and was removed once the graft took which was 1 to 2 wks. The date of when the graft took was recorded.

### TYLCV Inoculation Using Whiteflies.

In greenhouse studies, plants were inoculated with TYLCV using 5 adult whiteflies per plant added twice 1 week apart. The inoculation period lasted 2 weeks. Whiteflies were collected from TYLCV infected ‘Fl. Lanai’ tomato plants. Whiteflies were shaken off the plants onto yellow plastic cards and manually aspirated off the cards. In the graft studies, when inoculation occurred after the grafting, 1 clip cage per plant was used with 25 whiteflies in each cage, collected as above, added one time lasting for 1 wk. Inoculation period was terminated by ADMIRE (Bayer CropScience LP, Research Triangle Park, N.C.) except for the plants that were to be re-challenged with virus in which case they were treated with FULFILL (Syngenta Crop Protection, Inc., Greensboro, N.C.).

### TYLCV Symptom Rating.

Symptoms for TYLCV were rated on a scale of 0-4, where 0=no visible TYLCV symptoms, plants have same appearance as non-infected plants; 1=very slight yellowing of leaflet margins on apical leaf with minor downward leaf curling (DLC); 2=some yellowing, minor curling of leaflet ends and minor reduced leaf size (RLS); 3=a wide range of leaf yellowing, mild chlorosis at leaf margins, DLC, and cupping of leaflets, with some RLS, yet plants continue to develop; 4=full TYLCV symptoms; DLC, RLS, severe chlorotic margins, and stunting of plant (Lapidot and Friedman, 2002). In the case of grafting, symptom ratings of 0 to 1.0 are considered asymptomatic since plants often show very mild virus-like symptoms (1.0) due to age and the stress of grafting.

Following are examples which illustrate procedures for practicing the invention. These examples should not be construed as limiting. All percentages are by weight and all solvent mixture proportions are by volume unless otherwise noted.

### EXAMPLE 1

### Determining if Transgenic Plants are Immune

Before grafting, plants of line 67-10 and 45-10 were inoculated with TYLCV using whiteflies. The non-transgenic Fla. 7613 plants were not inoculated with TYLCV before grafting. Plants were grafted by the procedure described above 2 to 4 wks after inoculation using the following combinations: inoculated transgenic stocks (45-10 or 67-10) were grafted with scions with non-inoculated Fla. 7613 (scions). Controls were as follows: Fla. 7613 inoculated scions were grafted onto Fla. 7613 non-inoculated stocks and Fla. 7613 non-inoculated scions were grafted onto Fla. 7613 inoculated stocks. TYLCV symptom expression and the presence of TYLCV were determined in scions and stocks at 4, 8 and 12 wks after the graft was established.

Results: At 4 wks after establishment of the graft there were fewer plants showing infection of TYLCV than at 8 wks. There were no infected plants present in the grafts where the stock was susceptible and the scion was the inoculated transgenic line (45-10). There were 2 plants infected of those plants where the stock was the inoculated transgenic line and the scion was susceptible. In the experiments conducted with 67-10 there were more plants infected at 8 wks than at 4 wks and symptom expression was greater at 8 wks than at 4 wks. Therefore, the data for 4 wks is not shown and the data at 8 wks is presented in Table 1.

At 8 wks after establishment of the graft, there were only 2 plants out of 10 in which the susceptible stock which had been grafted with an inoculated transgenic (45-10) scion had become infected. This lack of resistance in 2 plants is consistent with frequency of resistance expected in this line. All other susceptible stocks were negative for TYLCV both by symptom expression and by PCR. In the ten plants where the stock was the inoculated transgenic plant (45-10) and the scion was a non-inoculated susceptible plant there were 3 plants in which the scion and the stock were infected with TYLCV as determined by both symptom expression and PCR. Symptoms in these plants were typical of TYLCV and were equivalent to the controls. All other plants grafted with this combination did not show any evidence of infection by TYLCV, symptom expression in these plants was rated 0, and PCR did not detect any TYLCV DNA. In comparison, 8 wks after the establishment of the grafts, all susceptible scions became infected after grafting onto inoculated susceptible scions, and all susceptible stocks became infected after grafting with inoculated susceptible scions in the control graft combinations. TYLCV DNA was detected in all susceptible scions and stocks. Symptom expression averaged 2.7 to 3.9 among the control graft combinations.

Nearly identical results were obtained using transgenic line 67-10. Transgenic stocks and scions in which no virus was detected, were not able to infect the susceptible scions and stocks grafted onto them. If no resistance was found in the transgenic stock or scion, then the susceptible tissue grafted onto it became infected, as evidence by symptom expression and PCR.

These results demonstrate that no virus was able to move out of inoculated transgenic plants into the susceptible lines in those transgenic plants that were resistant. This is consistent with the lack of detection of viral DNA in these plants after inoculation. This suggests that there is either no viral replication or extremely limited replication with no cell to cell (since there was no movement from transgenic tissue to susceptible tissue at the graft) or systemic movement of virus. Since the gene targeted by the transgene is the Rep gene, the former possibility is the more likely.

### EXAMPLE 2

### Inoculation of Transgenic Plants by Grafting of Infected Susceptible Plants

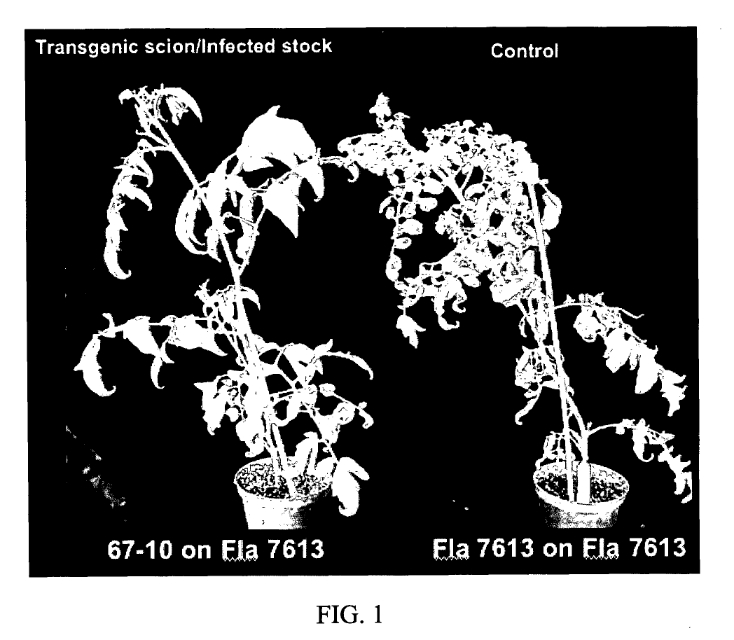

Before grafting, 32 plants for the Fla. 7613 line planted were inoculated with TYLCV. Plants were grafted by the procedure described above at 2 to 4 wks after inoculation using the following combinations: non-inoculated transgenic scions were grafted onto Fla. 7613 inoculated stocks, Fla. 7613 inoculated scions were grafted onto non-inoculated transgenic stocks (FIG. 1). Controls were as follows: Fla. 7613 inoculated scions were grafted onto Fla. 7613 non-inoculated stocks and Fla. 7613 non-inoculated scions were grafted onto Fla. 7613 inoculated stocks. Scions and stocks were assayed for the presence of TYLCV using symptom expression and PCR at 4 and 8 wks after the establishment of the grafts.

Results: Data is shown in Table 2. These studies were conducted with the transgenic line 67-10. A second study with line 45-10 is in progress.

There were only minor differences between the data at 4 and 8 wks after establishment of the grafts. Symptoms at 8 wks were slightly more developed, and a few more plants were infected. Therefore the data from 8 wks after establishment of the graft is shown.

At 8 wks after establishment of the graft, no to very mild symptoms of TYLCV were observed in resistant transgenic scions (mean of 1.4) and resistant transgenic stocks (mean of 1.3; Table 7). However, DNA was detected in all resistant transgenic scions and stocks. This is in contrast to non-grafted resistant transgenic in which no DNA is detected 3 wks to 2 months after inoculation. Eight wks after establishment of the graft in the controls, all susceptible and non-resistant transgenic plant parts (either stocks or scions) were infected with TYLCV, as evidenced by PCR, and all showed typical symptoms (means of 2.7 to 3.9).

Transgenic scions and stocks did not show symptoms of TYLCV infection like the susceptible controls but viral DNA was detected in them. The discrepancy between the lack of symptoms and the presence of DNA in transgenic plant tissues could be explained by an absence of replication of virus in the transgenic tissue but with a detection of viral DNA due to the systemic movement of viral DNA from the infected susceptible stocks or scions.

### EXAMPLE 3

### Determining if Resistance can be Translocated Across a Graft

Transgenic R₂ generation plants from line 45-10 and the non-transformed Fla. 7613, were used for this study. Before grafting, 64 plants of line 45-10 were inoculated with TYLCV as described. The non-transgenic Fla. 7613 plants were not inoculated with TYLCV before grafting. Two to 4 weeks after inoculation plants were grafted by the procedure described above in the following combinations: 10 transgenic 45-10 inoculated scions were grafted onto 10 Fla. 7613 non-inoculated stocks and 6 Fla. 7613 non-inoculated scions were grafted onto 6 transgenic 45-10 inoculated stocks. All the transgenic plants were negative for TYLCV as determined by PCR before grafting. Controls were as follows: 6 Fla. 7613 non-inoculated scions were grafted onto 6 Fla. 7613 non-inoculated stocks; Fla. 7613 non-inoculated scions onto inoculated Fla. 7613 stocks and inoculated Fla. 7613 scions onto non-inoculated Fla. 7613 stocks, Fla. 7613 grated onto 3 transgenic stocks that were not resistant to TYLCV. At 8 weeks after the graft was established TYLCV symptom expression was recorded and samples from all scions and stocks were assayed by PCR for the presence of TYLCV. At 8 wks after establishment of the graft, all the Fla. 7613 scions and stocks that were negative for TYLCV, were re-challenged by inoculation with TYLCV using clip cages (1 per inoculation) containing 25 whiteflies per clip cage. Two and 4 wks after re-challenge with TYLCV, plants were rated for symptom expression and were assayed by PCR for TYLCV.

Results: At 4 weeks post re-challenge, susceptible scions (Fla 7613) were uniformly infected with and displayed somewhat milder (3.0) symptoms of TYLCV than those of the controls (4.0) (Table 8). Susceptible scions that were grafted on 3 transgenic stock plants that were not resistant were uniformly infected with TYLCV and symptoms were identical to controls (4.0). In addition, TYLCV could now be detected in 4 of the 6 resistant transgenic stocks, although symptoms were not expressed in the transgenic stocks. There were 14 wks between the first challenge of the transgenic lines and the re-challenge of the susceptible line grafted onto them. Under these conditions, susceptible scions grafted onto challenged and resistant transgenic plants were not protected from inoculation by TYLCV. This implies that there was not a translocatable resistance factor present 8 weeks after an initial challenge.

In the reverse scenario, TYLCV DNA was detected in all re-challenged susceptible stocks and the symptom expression in these stocks was equivalent to those of the controls (3.8 vs 4.0). TYLCV DNA was detected in 7 out of 10 of the transgenic scions, however symptom expression was very mild to non-existent. Some plants in which there was no TYLCV DNA detected were rated as 1 or 2. This was probably due to the stress of grafting and age of the plant on the shape and color of the leaves.

### EXAMPLE 4

### Determining if Resistance can Overcome Previously Established Infection

Transgenic R₂ generation plants from line 45-10 and the non-transgenic parent line (Fla. 7613) were used for this study. Before grafting, 32 plants of both transgenic and non-transgenic lines were inoculated with TYLCV as described. The following grafts were made: inoculated transgenic plants (scions) were grafted onto inoculated Fla. 7613 stocks, and inoculated Fla. 7613 cuttings (scions) were grafted onto 45-10 stocks. Controls were as follows: Fla. 7613 inoculated scions on Fla. 7613 inoculated stocks, inoculated non-resistant 45-10 plants were grafted onto inoculated Fla. 7613 stocks, and inoculated Fla. 7613 scions were grafted onto inoculated non-resistant 45-10 stocks. Symptom expression was recorded and PCR assays for TYLCV were conducted 4, 8, 12, and 16 wks after the establishment of the graft.

Results: Inoculated transgenic stocks reduced the symptoms expressed in the susceptible scions at 4 wks after establishment of the grafts (Table 8). Inoculated susceptible scions grafted onto inoculated susceptible stocks had symptoms rated with a mean of 4.0, while inoculated susceptible scions grafted onto inoculated transgenic stocks expressed symptoms with a mean of 1.5. The symptom reduction remained relatively constant over time. The mean symptom rating of the scions was 1.8 at 8 wks after graft establishment, 1.0 at 12 wks, and 1.0 at 16 wks. Non-grafted susceptible plants would be expected to have symptoms of 3.0 to 4.0. A less significant reduction in symptom expression was observed when the scion was the transgenic genotype and the stock was the susceptible genotype. The inoculated susceptible stocks which were rated a 4.0 before grafting were rated as 3.2. This is only slightly reduced from the rating of 4.0 for the controls. These data suggest that a resistance factor was preferentially translocated up the plant, but was less efficiently translocated down the plant. This unidirectional movement implies the use of the phloem transport system for the resistance factor. More importantly, these data demonstrate that infected susceptible tissue can be cured of virus symptoms.

At 8 wks after the establishment of the graft, the symptoms on the susceptible scions were almost unchanged (mean symptom rating of 1.8). The symptoms on the susceptible stocks were slightly lower (mean symptom rating of 2.7) but basically unchanged. The symptoms on the transgenic scion were reduced (mean symptom rating of 0.6) while there were still no symptoms on the transgenic stock. Symptom expression in the susceptible scion was reduced to a mean of 1.0 by 12 weeks after grafting and remained unchanged by 16 weeks. The symptom on the other stocks and scions remained the same at 12 and 16 weeks after establishment of the graft.

These data suggest that 1) there is a translocatable resistance factor produced in challenged transgenic plants, 2) this factor is translocated preferentially up the plant, although there was some movement down the plant, 3) this resistance factor was able to interfere with an established infection and cause a reduction in symptom expression from full symptom expression to almost none.

### EXAMPLE 5

### Determining if Transgenic Stocks can Protect Susceptible Scions Without Prior Challenge

Transgenic R₂ generation plants from line 45-10 and 67-10, and the non-transgenic parent line (Fla. 7613) were used for this study. Non-inoculated transgenic stocks were grafted to non-inoculated non-transgenic scions and the reverse was also done (transgenic scions grafted onto non-transgenic stocks). These experiments were conducted twice, once in cooler temperatures of winter and once in higher temperatures of late spring. This is because symptom expression is usually milder in winter when light intensity and temperatures are lower, and symptom expression is greater when temperature and light intensity increase. Once grafts were established, plants were inoculated with whiteflies which had been reared on TYLCV-infected tomatoes. The inoculation period was 5 days. Plants were treated with Admire to end the inoculation period. Symptoms were read 4 and 8 weeks after the end of the inoculation period. Plants were sampled at the time visual assessments were made and tested for the presence of viral DNA using PCR.

Results: Susceptible scions became infected after inoculation but within weeks symptom severity began to lessen. Presumably this was due to the turning on of the resistance factor in the transgenic stock, which was then translocated to the scions where the established infection was turned off. Results from the winter trial are shown in Table 5, those of the spring study in Table 6. These results are consistent with the results of Examples 1-4. Not all transgenic plants acted the same; the 45-10 line was superior to line 67-10 in its ability to reduce symptoms in susceptible scions. Thus, transgenic plants can be screened for those that are able to perform the best as stocks for eliminating virus from susceptible scions. This also suggests that transgenic lines could be found which reduce symptoms (virus) from susceptible scions more effectively than is demonstrated in these studies. There was a significant reduction in the mean severity of symptoms produced in susceptible and inoculated scions. Fla 7613 grafted onto Fla 7613 had a mean symptom severity of 3.75, compared to Fla 7613 grafted onto line 45-10, with a mean symptom severity of 2.26 or Fla 7613 grafted onto 67-10 which had a mean symptom severity of 3.10. The reduction in symptoms is 40% and 17% respectively. Reduction in symptom severity is directly related to reduction in yields in tomato. If resistance was uniformly present in the transgenic plants being studied (R₂ generation plants are still segregating for resistance), the effects of the transgenic stocks would be more pronounced.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

TABLE 1
Determining if resistant transgenic plants are immune to infection by TYLCV by
grafting of susceptible non-inoculated tissues onto inoculated resistant transgenic plant
tissues

| | | | | | STOCK | | SCION | |
|---|---|---|---|---|---|---|---|---|
| | | | | | NO. | MEAN | NO. | MEAN |
| STOCK | | SCION | | NO. of | RESIST. | SYMPT. | RESIST. | SYMPT. |
| IDENTITY | TREATMENT | IDENTITY | TREATMENT | GRAFTS | STOCKS | RATING | SCIONS | RATING² |
| 45-10 | Inoc. before | Fla.7613 | NI | 7 | 7 | 0 | 7 | 0 |
| | grafting | | | | | | | |
| 67-10 | Inoc. before | Fla.7613 | NI | 8 | 8 | 0.6 | 8 | 0.4 |
| | grafting | | | | | | | |
| Fla.7613 | Inoc. before | Fla.7613 | NI | 10 | 0 | 3.1 | 0 | 2.7 |
| | grafting | | | | | | | |
| Fla.7613 | NI | Fla.7613 | Inoc. before | 8 | 0 | 3.9 | 0 | 3.3 |
| | | | grafting | | | | | |
| Fla.7613 | NI | 67-10 | Inoc. before | 4 | 0 | 4.0 | 0 | 4.0 |
| | | NR³ | grafting | | | | | |

¹NI = Not inoculated.
²Symptom rating of infected plants. Symptom rating of resistant transgenic and non-transgenic plants ranged from a mean of 0.0 to 0.7.

TABLE 2
Inoculation of resistant transgenic stocks and scions by grafting with TYLCV-
infected susceptible scions or stocks. Data shown was collected at 8 weeks after
establishment of the graft; data in parenthesis was collected at 12 weeks after
establishment of the graft.

| | | | | | STOCK | | SCION | |
|---|---|---|---|---|---|---|---|---|
| | | | | | NO. | MEAN | NO. | MEAN |
| STOCK | | SCION | | NO. of | RESIST. | SYMPT. | RESIST. | SYMPT. |
| IDENTITY | TREATMENT | IDENTITY | TREATMENT | GRAFTS | STOCKS | RATING | SCIONS | RATING |
| 02-09 | NI | Fla.7324 | Inoc. before | 3 | 2 | NR | 1 | NR |
| | | | grafting | | | | | |
| 45-10 | NI | Fla.7613 | Inoc. before | 10 | 0 | 1.2 | 0 | 3.7 |
| | | | grafting | | | | | |
| 67-10 | NI | Fla.7613 | Inoc. before | 10 | 0 | 2.1 | 1 | 1.5 |
| | | | graft | | | | | |
| Fla.7324 | Inoc. | 02-09 | NI | 7 | 0 | NR | 0 | NR |
| | before graft | | | | | | | |
| Fla.7613 | Inoc. | 45-10 | NI | 10 | 0 (0) | 4.0 (3.0) | 0 (2) | 2.9 (1.6) |
| | before graft | | | | | | | |
| Fla.7613 | Inoc. | 67-10 | NI | 9 | 0 | 1.3 | 0 | 3.4 |
| | before graft | | | | | | | |
| Fla.7613 | Inoc. | Fla.7613 | NI | 10 | 0 | 3.1 | 0 | 2.7 |
| | before graft | | | | | | | |
| Fla.7613 | NI | Fla.7613 | Inoc. before | 8 | 0 | 3.9 | 0 | 3.3 |
| | | | graft | | | | | |

NI = not inoculated
NR = Not rated for symptoms

TABLE 3
Translocation of a resistance factor across a graft from plants transformed with
2/5 Rep and inoculated with TYLCV.

| | | | | | STOCK | | SCION | |
|---|---|---|---|---|---|---|---|---|
| | | | | | NO. | MEAN | NO. | MEAN |
| STOCK | | SCION | | NO. of | RESIST. | SYMPT. | RESIST. | SYMPT. |
| IDENTITY | TREATMENT | IDENTITY | TREATMENT | GRAFTS | STOCKS | RATING¹ | SCIONS | RATING¹ |
| Fla7613 | Inoc. after | 45-10 | Inoc. before | 10 | 0 | 3.8 | 3 | 0.9² |
| | grafting | | grafting | | | | | |
| 45-10 | Inoc. | Fla7613 | Inoc. after | 6 | 2 | 0.3 | 0 | 3.0 |
| | before | | grafting | | | | | |
| | grafting | | | | | | | |
| Fla7613 | Inoc. after | Fla7613 | Non-inoc. | 3 | 0 | 4.0 | 0 | 2.3 |
| | grafting | | | | | | | |
| Fla7613 | Non-inoc. | Fla7613 | Inoc. after | 3 | 0 | 4.0 | 0 | 4.0 |
| | | | grafting | | | | | |
| 45-10 | Inoc. | Fla7613 | Inoc. after | 3 | 0 | 4.0 | 0 | 4.0 |
| NR³ | before | | grafting | | | | | |
| | graft | | | | | | | |

¹Mean symptom rating of infected plants.
²Mean symptom rating of PCR negative plants was 1.3.

TABLE 4
Non-transgenic and transgenic plants inoculated with TYLCV and
then grafted to each other.

| Stock | Scion | 0 | 4 | 8 | 12 |
|---|---|---|---|---|---|
| | | MEAN SYMPTOM RATING OF THE SCION | | | |
| | | No. weeks after graft establishment | | | |
| 45-10 | Fla 7613 | 4.0 | 1.5 | 1.8 | 1.0 |
| Fla 7613 | 45-10 | 1.3 | 1.2 | 0.6 | 0.4 |
| Fla 7613 | Fla 7613 | 4.0 | 4.0 | 4.0 | 4.0 |
| | | MEAN SYMPTOM RATING OF THE STOCK | | | |
| | | No. weeks after graft establishment | | | |
| 45-10 | Fla 7613 | 0.1 | 0.1 | 0.0 | 0.0 |
| Fla 7613 | 45-10 | 3.2 | 3.2 | 2.7 | 2.2 |
| Fla 7613 | Fla 7613 | 4.0 | 4.0 | 4.0 | 4.0 |

¹Mean symptom rating of infected plants.
²Mean symptom rating of PCR negative plants was 0.3
³NI = Non-inoculated.

TABLE 5
Transgenic and susceptible grafted plants inoculated with TYLCV after graft
establishment during winter season 2003. Infection rate of whole plants inoculated after
establishment of the graft, infection based on symptom expression. Data shown is at 12
weeks post inoculation.

| | | | | Stock | | Scion | |
|---|---|---|---|---|---|---|---|
| | | | % | | No. of | | No. |
| | | | Infection | No. of Plants | Plants | No. plants | plants |
| | | No. of | in | Without | With | Without | With |
| Stock | Scion | Plants | Scions | symptoms¹ | symptoms | symptoms | symptoms² |
| 45-10 | 7613 | 16 | 12.5 | 16 (0.5) | 0 | 14 (1.3) | 2 (3.5) |
| 67-10 | 7613 | 18 | 22.2 | 18 (0.4) | 0 | 14 (0.7) | 4 (2.9) |
| 7613 | 45-10 | 19 | 6.3 | 18 (1.6) | 1 (2.5) | 18 (0.6) | 1 (3.0) |
| 7613 | 67-10 | 18 | 0 | 18 (0.9) | 0 | 18 (0.1) | 0 |
| 45-10 | 45-10 | 6 | 0 | 6 (0.5) | 0 | 6 (0.5) | 0 |
| 67-10 | 67-10 | 6 | 0 | 6 (0.5) | 0 | 6 (0.5) | 0 |
| 7613 | 7613 | 6 | 100 | 0 | 6 (2.2) | 0 | 6 (2.6) |
| 7613 | Not | 6 | 100 | 0 | 6 (2.5) | | |
| | grafted | | | | | | |

¹Value in bold parentheses is the mean symptom rating of the plants.
²Some plants are susceptible due to the presence of segregation for resistance in the transgenic plants.

TABLE 6
Non-transgenic and transgenic tomato plants grafted and then inoculated
with TYLCV during the spring 2004.

| | | MEAN SYMPTOM RATING OF THE SCION¹ | | | |
|---|---|---|---|---|---|
| | | (wk after inoculation) | | | |
| Stock | Scion | 0 | 4 | 8 | 12² |
| 45-10 | Fla 7613 | 0.1 | 3.67 | 2.26 | |
| Fla 7613 | 45-10 | 0.0 | 1.48 | 1.29 | |
| 67-10 | Fla 7613 | 0.0 | 3.90 | 3.10 | |
| Fla 7613 | 67-10 | 0.0 | 2.27 | 0.94 | |
| Fla 7613 | Fla 7613 | 0.0 | 3.0 | 3.75 | |

¹Means include those of some non-resistant transgenic plants (due to presence of segregation for resistance)
²This experiment is still in progress.

TABLE 7
Can Transgenic Plants be Infected by Graft Inoculation with Infected
Plants?

| | Scion (not | Symptom Rating | | |
|---|---|---|---|---|
| Stock (Inoc. | inoc. before | in the Scion | | |
| Before grafting) | grafting) | 4 | 8 | 12 |
| Fla 7613 | 67-10 | 1.1 | 1.5 | 1.2* |
| Fla 7613 | Fla 7613 | 3.7 | 3.8 | 3.8 |

Symptom Expression (0-4 scale; Lapidot)
*PCR detection: positive, but negative after removal from the plant

TABLE 8

| STOCK | SCION | | | | | |
|---|---|---|---|---|---|---|
| (Inoc. | (Inoc. | | | | | |
| before | before | | | | | |
| graft) | graft) | 0 | 4 | 8 | 12 | |
| | | SYMPTOM RATING IN THE SCION | | | | |
| | | (wk after graft establishment) | | | | |
| 45-10 | Fla 7613 | 4.0 | 1.5 | 1.8 | 1.0 | [symbol] |
| Fla 7613 | 45-10 | 1.3 | 1.2 | 0.6 | 0.4 | |
| Fla 7613 | Fla 7613 | 4.0 | 4.0 | 4.0 | 4.0 | |
| | | SYMPTOM RATING IN THE STOCK | | | | |
| | | (wk after graft establishment) | | | | |
| 45-10 | Fla 7613 | 0.1 | 0.1 | 0.0 | 0.0 | [symbol] |
| Fla 7613 | 45-10 | 3.2 | 3.2 | 2.7 | 2.2 | |
| Fla 7613 | Fla 7613 | 4.0 | 4.0 | 4.0 | 4.0 | |

### REFERENCES

U.S. Pat. No. 6,818,804
U.S. Pat. No. 6,777,588
U.S. Pat. No. 6,750,382
U.S. Pat. No. 6,716,967
U.S. Pat. No. 6,667,426
U.S. Pat. No. 6,172,280
U.S. Pat. No. 6,852,907
U.S. Pat. No. 6,548,742
U.S. Pat. No. 6,057,492
U.S. Pat. No. 6,127,601
U.S. Pat. No. 5,530,193
U.S. Pat. No. 5,998,699
Worldwide Website: www.agnet.org/library/article/eb480.html
Worldwide Website: www.paramount-seeds.com/Paramountonline/grafting.htm.
Altschul et al. (1990) J. Mol. Biol. 215:402-410.
Altschul et al. (1997) Nucl. Acids Res. 25:3389-3402.
Barajas, D. et al. (2004) “Resistance to Plum pox virus (PPV) in Nicotiana benthalmana plants transformed with the PPVHC-PRO silencing suppressor gene” Journal of Plant Pathology 86(3):239-248.
Beltz, G. A., K. A. Jacobs, T. H. Eickbush, P. T. Cherbas, and F. C. Kafatos (1983) Methods of Enzymology, R. Wu, L. Grossman and K. Moldave [eds.] Academic Press, New York 100:266-285.
Black, L. L. et al. (2003) “Grafting Tomatoes for Production in the Hot-Wet Season” International Cooperators' Guide Asian Vegetable Research & Development Center Publication No. 03-551, pp. 1-6.
Edelstein, M. et al. (2004) “Assessing the effect of genetic and anatomic variation of Cucurbita rootstocks on vigour, survival and yield of grafted melons” Journal of Horticultural Science & Biotechnology 79(3):370-374.
Fernandez-Garcia et al. (2004) “Fruit quality of grafted tomato plants grown under saline conditions” Journal of Horticultural Science & Biotechnology 79(6):995-1001.
Karlin and Altschul (1990) Proc. Natl. Acad. Sci. USA 87:2264-2268.
Karlin and Altschul (1993) Proc. Natl. Acad. Sci. USA 90:5873-5877.
Lanfermeijer, F. C. et al. (2004) “The durable resistance gene Tm-2(2) from tomato conifers resistance against ToMV in tobacco and preserves its viral specificity” Plant Science 167(4):687-692.
Lewin, B. (1985) Genes II, John Wiley & Sons, Inc., p. 96.
Maniatis, T., E. F. Fritsch, J. Sambrook (1982) Molecular Cloning: A Laboratory Manual, Cold Spring Harbor Laboratory, Cold Spring Harbor, N.Y.
Yang, Y. et al. (2004) “Use of Tomato Yellow Leaf Curl Virus (TYLCV) Rep Gene Sequences to Engineer TYLCV Resistance in Tomato” Phytopathology 94(5):490-496.

1. A plant comprising a transformed or transgenic rootstock that is resistant to a plant pathogen, and grafted onto said transformed or transgenic rootstock, a non-transgenic plant tissue that is compatible with said rootstock.

2. The plant according to claim 1, wherein said transformed or transgenic rootstock exhibits resistance to infection by tomato yellow leaf curl virus (TYLCV).

3. The plant according to claim 1, wherein said non-transgenic plant tissue is a scion from a non-transgenic, TYLCV-susceptible plant.

4. The plant according to claim 1, wherein said non-transgenic plant tissue is a scion.

5. The plant according to claim 1, any preceding claim, wherein said plant pathogen is a virus, viroid, bacteria, insect, or fungus.

6. The plant according to claim 5, wherein said virus is selected from the group consisting of tobacco mosaic virus, cucumber mosaic virus, ringspot virus, necrosis virus, maize dwarf mosaic virus, tomato yellow leaf curl virus, tomato mottle virus, soybean mosaic virus, tomato spotted wilt virus, barley yellow dwarf virus, citrus tristeza virus, and citrus mosaic virus.

7. The plant according to claim 5, wherein said bacteria is Xanthomonas axonopodis pv. citri.

8. The plant according to claim 1, wherein said plant is a dicotyledonous plant.

9. The plant according to claim 8, wherein said dicotyledonous plant is selected from the group consisting of peas, alfalfa, tomato, melon, chickpea, chicory, clove, kale, lentil, soybean, tobacco, potato, sweet potato, radish, cabbage, rape, apple trees, grape, cotton, sunflower, citrus, and lettuce.

10. The plant according to claim 9, wherein the citrus is selected from the group consisting of orange, mandarin, kumquat, lemon, lime, grapefruit, tangerine, tangelo, citron, and pomelo.

11-13. (canceled)

14. The plant according to claim 1, wherein said transformed or transgenic rootstock comprises a polynucleotide comprising a nucleotide sequence selected from the group consisting of SEQ ID NO: 1, SEQ ID NO: 2, and SEQ ID NO: 3.

15. A method for producing a plant having increased or improved disease and/or pathogen resistance, wherein said method comprises grafting a transformed or transgenic rootstock of a plant having resistance to a plant pathogen onto a compatible non-transgenic plant tissue.

16. The method according to claim 15, wherein said transformed or transgenic rootstock exhibits resistance to infection by tomato yellow leaf curl virus (TYLCV).

17. The method according to claim 15, wherein said non-transgenic plant tissue is a scion from a non-transgenic, TYLCV-susceptible plant.

18. The method according to claim 15, wherein said non-transgenic plant tissue is a scion.

19. The method according to claim 15, wherein said plant pathogen is a virus, viroid, bacteria, insect, or fungus.

20. The method according to claim 19, wherein said virus is selected from the group consisting of tobacco mosaic virus, cucumber mosaic virus, ringspot virus, necrosis virus, maize dwarf mosaic virus, tomato yellow leaf curl virus, tomato mottle virus, soybean mosaic virus, tomato spotted wilt virus, barley yellow dwarf virus, citrus tristeza virus, and citrus mosaic virus.

21. The method according to claim 19, wherein said bacteria is Xanthomonas axonopodis pv. citri.

22. The method according to claim 15, wherein said plant is a dicotyledonous plant.

23. The method according to claim 22, wherein said dicotyledonous plant is selected from the group consisting of peas, alfalfa, tomato, melon, chickpea, chicory, clove, kale, lentil, soybean, tobacco, potato, sweet potato, radish, cabbage, rape, apple trees, grape, cotton, sunflower, citrus, and lettuce.

24. The method according to claim 23, wherein the citrus is selected from the group consisting of orange, mandarin, kumquat, lemon, lime, grapefruit, tangerine, tangelo, citron, and pomelo.

25-27. (canceled)

28. The method according to claim 15, wherein said transformed or transgenic rootstock comprises a polynucleotide comprising a nucleotide sequence selected from the group consisting of SEQ ID NO: 1, SEQ ID NO: 2, and SEQ ID NO: 3.

29. A fruit produced by a plant as defined in claim 1.


SEQUENCE LISTING (3 sequences)

SEQ ID NO: 1 - DNA, 506 bp, tomato yellow leaf curl virus
gccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatcc

SEQ ID NO: 2 - DNA, 1871 bp, tomato yellow leaf curl virus
gccattaggtgtcgaggtattagtaagacaccgatacaccgattgccatagagctttgagggacaccgattcatttcaacatgcctcgtttatttaaaatatatgccaagaattatttcctaacatatcccaattgttctctctctaaagaggaagcactttcccaattaaaaaacatagaaaccccaacaaataaaaaatacatcaaagtttgcagagaattccacgagaatggggaaccacatctccatgtgcttatccaattcgaaggcaaataccaatgtaagaaccaacggttcttcgacctggtatccccaaacaggtcagcacatttccatccaaacattcaggcagctaagagctcaacagatgtcaagacctacgtggagaaagacggagacttcattgattttggagttttccaaatcgatggcagatcagctagaggaggtcagcaatctgccaacgacgcatacgccggagcactcaattcaggcagtaaatccgaggccctcaatatattaaaagaaaaggccccaaaggactatattttacaatttcataatttaaattcaaatttagataggatttttagtcctcctttagaagtttatgtttctccatttctttcttcttcttttaatcaagttccagatgaacttgaagagtgggtcgccgagaacgtcgtgtcttccgctgcgcggccatggagacccataagtattgtcattgagggtgatagcagaacaggcaaaacaatgtgggccaggtctctaggcccacataattatttatgtggacatctagacctaagcccaaaggtgtacagtaatgatgcgtggtacaacgtcattgatgacgtagacccgcattatttaaagcacttcaaggaattcatgggggcccagagggactggcaaagcaacacaaagtacgggaagcccattcaaattaaagggggaattcccactatcttcctctgcaatccaggacctacctcctcatatagggaatatctagacgaagaaaaaaacatatccttgaaaaattgggctctcaagaatgcaaccttcgtcaccctctacgagccactgttcgcaagtatcaatcaaggtccaacacaagatagccaagaagaaaccaataaggcgtaagcgtgtagacctagactgtggctgctcatactacgagcgacccactcttcaagttcatctggaacttgattaaaagaagaagaaagaaatggagaaacataaacttctaaaggaggactaaaaatcctatctaaatttgaatttaaattatgaaattgtaaaatatagtcctttggggccttttcttttaatatattgagggcctcggatttactgcctgaattgagtgctccggcgtatgcgtcgttggcagattgctgacctcctctagctgatctgccatcgatttggaaaactccaaaatcaatgaagtctccgtctttctccacgtaggtcttgacatctgttgagctcttagctgcctgaatgtttggatggaaatgtgctgacctgtttggggataccaggtcgaagaaccgttggttcttacattggtatttgccttcgaattggataagcacatggagatgtggttccccattctcgtggaattctctgcaaactttgatgtattttttatttgttggggtttctatgttttttaattgggaaagtgcttcctctttagagagagaacaattgggatatgttaggaaataattcttggcatatattttaaataaacgaggcatgttgaaatgaatcggtgtccctcaaagctctatggcaatcggtgtatcggtgtcttactaatacctcgacacctaatggcaattt

SEQ ID NO: 3 - DNA, 681 bp, tomato yellow leaf curl virus
gcgacccactcttcaagttcatctggaacttgattaaaagaagaagaaagaaatggagaaacataaacttctaaaggaggactaaaaatcctatctaaatttgaatttaaattatgaaattgtaaaatatagtcctttggggccttttcttttaatatattgagggcctcggatttactgcctgaattgagtgctccggcgtatgcgtcgttggcagattgctgacctcctctagctgatctgccatcgatttggaaaactccaaaatcaatgaagtctccgtctttctccacgtaggtcttgacatctgttgagctcttagctgcctgaatgtttggatggaaatgtgctgacctgtttggggataccaggtcgaagaaccgttggttcttacattggtatttgccttcgaattggataagcacatggagatgtggttccccattctcgtggaattctctgcaaactttgatgtattttttatttgttggggtttctatgttttttaattgggaaagtgcttcctctttagagagagaacaattgggatatgttaggaaataattcttggcatatattttaaataaacgaggcatgttgaaatgaatcggtgtccctcaaagctctatggcaatcggtgtatcggtgtcttactaatacctcgacacctaatggcaattt